(19) United States
(12) Patent Application Publication
(10) Pub. No.: US 2025/0325190 A1
(43) Pub. Date: Oct. 23, 2025

(54) METHODS OF MEASURING FLUORESCENCE AND CAPTURING MOVEMENT IN ANIMALS

(71) Applicant: Georgia Tech Research Corporation, Atlanta, GA (US)

(72) Inventors: Jeffrey Markowitz, Atlanta, GA (US); Emine Zeynep Ulutas, Atlanta, GA (US)

(21) Appl. No.: 19/183,668

(22) Filed: Apr. 18, 2025

Related U.S. Application Data
(60) Provisional application No. 63/636,585, filed on Apr. 19, 2024.

(51) Int. Cl. A61B 5/00; A61K 9/00; A61K 49/00

(52) U.S. Cl. CPC A61B 5/0071; A61K 9/0019; A61K 49/0017; A61K 49/0067

(57) ABSTRACT

The present disclosure relates to methods of administering fluorescent particles to animal and for measuring the location of the fluorescent particles within an animal over time.

### CROSS-REFERENCE TO RELATED APPLICATION

This application claims the benefit of U.S. Provisional Application No. 63/636,585, filed Apr. 19, 2024, the entire contents of which are incorporated by reference herein.

### BACKGROUND

Quantifying movement plays an essential role in understanding how the brain coordinates action selection. Accurately capturing movement becomes particularly challenging in one of the most common laboratory animals, mice, where their behaviors unfold over milliseconds, and their fur and soft tissues significantly obscure movement of the skeleton. These issues are critical since the skeleton, and not the fur and soft tissues, is the part of the body actually controlled by the brain (Monsees et al., 2022). Most current methods for measuring movement in mice, including popular machine-learning based approaches (Mathis et al., 2018; Pereira et al., 2022), cannot directly measure skeletal dynamics. Given that the mouse is one of the most popular model systems in both basic and translational neuroscience, direct measurement of the skeleton in a freely moving mouse is critical for future studies.

### SUMMARY OF THE INVENTION

In one aspect, the present invention is directed to a method of measuring fluorescent particles within animals. In some embodiments of the present invention, the animal is injected with fluorescent particles, and the fluorescence is captured.

In some embodiments of the present invention, the fluorescent particle is a quantum dot.

In some embodiments of the present invention, the quantum dot emits at an excitation wavelength from about 700 nm to about 850 nm.

In some embodiments of the present invention, the animal is a mammal.

In some embodiments of the present invention, the mammal is a mouse.

In some embodiments of the present invention, the animal (such as a mouse) is injected in at least one of the right paw, the left paw, the right hind leg, the left hind leg, the tail. Exemplary non-limiting modes of injection include, but are not limited to, intramuscular, intradermal, subdermal, and spinal.

In some embodiments of the present invention, a camera captures the fluorescence. In some instances, the camera is an infrared camera.

In some embodiments of the present invention, the animal is a diseased animal. In some instances, the disease is a neurodegenerative disease. In some instances, the neurodegenerative disease is Parkinson's disease or Huntington's disease. In some instances, the disease is cancer. In some instances, the cancer is a blood cancer. In some instances, the cancer is a tumor.

### BRIEF DESCRIPTION OF DRAWINGS

FIG. 1 is an illustrative schematic of a technique for high-resolution, 3D tracking of fluorescent markers embedded under the skin in live mice.

FIG. 1A is a schematic of where NIR-fluorescent quantum dots (QDs) are injected in key location key locations in mice (FIG. 1A, left). They are then imaged using a cross-polarized and hardware-synchronized 5 camera system (Baslet Ace 2 NIR at 30 frames per second, FIG. 1A, right).

FIG. 1B is a graph illustrating how long pixel intensity can be measured in mice. The gray shaded area represents the 95% confidence interval of vehicle-injected controls (n=3 mice, n=5 views). Maximum per-frame fluorescence is measured, averaged over all frames, in QD-injected mice (blue shading indicates 1 standard deviation, n=3 mice, n=5 camera views per mouse, Qtracker™ 800 Cell Labeling kit from ThermoFisher) remains well-above detection threshold for up to 72 hours.

FIG. 1C shows images that are both reflectance (top) and fluorescent (bottom) frames that are collected through the same cameras using fast time-division multiplexing with separate light sources (730 nm and 850 nm, Advanced Illumination).

FIG. 2 is a picture of an exemplary staging arena used to capture images of mice injected with quantum dots.

### DETAILED DESCRIPTION OF THE INVENTION

Various aspects of the present invention pertain to a method of tracking fluorescent particles injected into an animal. In some instances, the animal is a mammal. In some instances, the mammal is a mouse. In some instances, the fluorescent particles are nanoparticles. In some instances, the fluorescent particles are quantum dots. In some instances, the fluorescent particles are nanospheres having a coating on an external surface thereof, the coating comprising one or more fluorescent dyes. In some instances, the fluorescent particles are microspheres having a coating on an external surface thereof, the coating comprising one or more fluorescent dyes. In some instances, the fluorescent particles are quantum dots having a coating on an external surface thereof, the coating comprising one or more fluorescent dyes. In some instances, the fluorescent particles are quantum dots that are intrinsically fluorescent. In some instances, the fluorescent particles are other micro or nano-particles coated with a fluorescent dye. In some instances, the fluorescent particles are latex particles have a coating on an external surface thereof, the coating comprising one or more fluorescent dyes. In some instances, the fluorescent particles are biocompatible particles have a coating on an external surface thereof, the coating comprising or more fluorescent dyes.

As used herein, the term “about” when appearing before a range should be understood as referring to both endpoints of the range. In such instances the range should also be understood as including the range defined by the specific endpoints listed, and also including sub-ranges within the listed endpoints. In the instances where “about’ is appearing before a number it should be understood as the number includes the range of +/−5%.

In accordance with various aspects of the disclosure, the fluorescent particles, such as quantum dots, are injected into an animal, such as a mouse. In some instances, the fluorescent particles, such as quantum dots, are injected into the left forepaw. In some instances, the fluorescent particles, such as quantum dots, are injected into the right forepaw. In some instances, the fluorescent particles, such as quantum dots, are injected into the right hind leg. In some instances, the fluorescent particles, such as quantum dots, are injected into the left hind leg. In some instances, the fluorescent particles, such as quantum dots, are injected along the spine. In some instances, the fluorescent particles, such as quantum dots, are injected into the tail. In some instances, the fluorescent particles, such as quantum dots, are injected subdermally in the animal. In some instances, more than one type of fluorescent particles, such as more than one type of quantum dot, is injected.

In accordance with various aspects of the disclosure, quantum dots are nanoparticles that fluoresce upon excitation by a light source having an excitation wavelength. In some instances, the excitation wavelength of the quantum dot ranges from about 600 nm to about 1000 nm. In some instances, the excitation wavelength of the quantum dot ranges from about 600 nm to about 1000 nm. In some instances, the excitation wavelength of the quantum dot ranges from about 680 nm to about 800 nm. In some instances, the excitation wavelength of the quantum dot ranges from about 700 nm to about 760 nm. If the excitation wavelength of the quantum dot dips below 600 nm, the excitation wavelength starts to fall into the visibility spectrum of the animal (such as a mouse). Therefore, the animal will start to see itself fluoresce. If the excitation wavelength of the quantum dot starts to go above 1000 nm, it starts to be absorbed by water within the animal preventing an accurate reading.

In accordance with various aspects of the disclosure, the quantum dot can be coated. In some instances, the quantum dot is coated with an amphiphilic polymer coating. In some instances, the coating is a carboxyl-derivatized amphiphilic coating. In some instances, the coating can be coupled to a peptide. In some instance the coating can be coupled to the amine group of proteins. In some instances, the coating can be coupled to modified oligonucleotides. In some instances, the coating is an aliphatic hydrocarbon surface coating. In some instances, the coating is polyethylene glycol (PEG). In some instances, the coating is streptavidin. In some instances, the quantum dot is the Qtracker™ 800 Cell Labeling kit from ThermoFisher. In some instances, the quantum dot is the Qtracker™ 800 Vascular label from ThermoFisher. In some instances, the quantum dot is the Qdot™ 800 Streptavidin Conjugate from ThermoFisher. In some instances, the quantum dot is a Qdot 800 Probe from ThermoFisher. In some instances, the quantum dot is labelled with an antibody. In some instances, the quantum dot is labelled using the Qtracker™ 800 Cell Labeling Kit.

In accordance with various aspect of the disclosure, after injection with quantum dots, the animal is placed in a staging area to measure fluorescent intensity of the quantum dot. In some instances, the fluorescent intensity is measurable from about 1 day to about 7 days post injection. In some instances, the fluorescent intensity is measurable for about 1 day post injection. In some instances, the fluorescent intensity is measurable for about 2 days post injection. In some instances, the fluorescent intensity is measurable for about 3 days post injection. In some instances, the fluorescent intensity is measurable for about 4 days post injection. In some instances, the fluorescent intensity is measurable for about 5 days post injection. In some instances, the fluorescent intensity is measurable for about 6 days post injection. In some instances, the fluorescent intensity is measurable for about 7 days post injection. In some instances, the fluorescent intensity is measurable for about 8 days post injection. In some instances, the fluorescent intensity is measurable for about 9 days post injection. In some instances, the fluorescent intensity is measurable for about 10 days post injection. In accordance with various aspects of the disclosure the fluorescence of the quantum dots is captured or recorded. In some instances, the fluorescence of the quantum dots is captured from multiple angles. In some instances, the animal is surrounded by devices that can capture the fluorescence emitting from the animal. The multiple angles allow an accurate 3D reconstruction of fluorescent key points under the animal's skin as they move around. In some instances, the cameras capture at least 10 frames of the moving fluorescent animal. In some instances, the cameras capture at least 100 frames of the moving fluorescent animal. In some instances, the cameras capture at least 1000 frames of the moving fluorescent animal. In some instances, the cameras capture at least 10000 frames of the moving fluorescent animal. In some instances, the cameras capture at least 100000 frames of the moving fluorescent animal. In some instances, the cameras capture at least 1000000 frames of the moving fluorescent animal. In some instances, the cameras capture at least 10000000 frames of the moving fluorescent animal. In some instances, the fluorescence can be tracked from using computer software. In some instances, the tracking computer software is SLEAP. In some instances, the tracking computer software is DeepCutLab. In some instances, the fluorescence of the quantum dots is captured through camera. In some instances, the camera is a near-infrared camera.

In accordance with various aspects of the present disclosure, the animal is placed in a recording arena. In some instances, the recording arena is made of plexiglass. In certain aspects of the present invention, the animal is injected with a quantum dot, and then has their fluorescence measured for one or more days.

In accordance with various aspects of the present disclosure, the animal to be injected in a diseased animal. In some instances, the diseases is cancer. In some instances, the cancer is a blood cancer. In some instances, the cancer is a solid tumor. In some instances, the disease is a neurodegenerative disorder. In some instances, the neurodegenerative disorder is Alzheimer's, dementia, Ataxia, Huntington's disease, Parkinson's disease, motor neuron disease, multiple system atrophy, or progressive supranuclear palsy. In some embodiments of the present invention, movement loss in neurodegenerative diseases is measured through injection of the quantum dots in the animal.

In accordance with various aspects of the present disclosure, the animal has their movement captured via measuring the fluorescent particles. In some instance the movement is captured to look for differences between diseased animals and healthy animals. In some instances, the gait is measured in the animals. In some instances, the animal in measured for a halting gait. In some instances, the animal is measured for a tremor. In some instances, the animal is measured for dyskinesia. In some instances, the dyskinesia is due to L-DOPA treatment. In some instances, the animal is measured for tics. In some instances, the animal is measured for chorea. In some instances, the movements measure, such as gait or tics, is compared between a diseased animal and a healthy animal.

### Examples

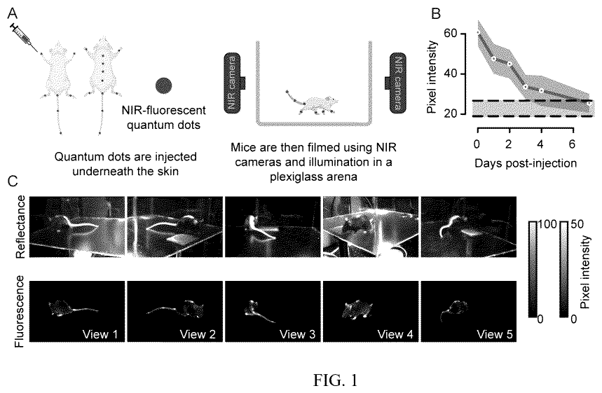

Fluorescent nanoparticles called “quantum dots” are utilized to visualize key points in vivo. Quantum dots are semiconductor nanoparticles that are highly photostable, have minimal photobleaching, and high quantum yield at wavelengths ranging from blue to near-infrared (NIR) (Reineck & Gibson, 2017). To noninvasively image through the fur and soft tissues, near-infrared (NIR) quantum dots may be used to image wavelengths within the NIR-I spectrum. Quantum dots are introduced into the body through injecting with a customized Drummond (Drummond Scientific, Cat. No. 3-000-510) and glass pipette. FIG. 1A, left, displays possible injection points in mice in the hands, feet, tail, and spine, for example. FIG. 1A, right, displays the mouse placed in the staging area surrounded by NIR cameras, wherein the red dots signify the injected quantum dots from FIG. 1A, left. QDot 800 vascular labels (ThermoFisher Scientific, Cat. No. Q21071MP) and the QDot 800 cell labeling kit (ThermoFisher Scientific, Cat. No. Q25071MP) have been tested measuring the NIR wavelengths. After injection, animals are put in an arena where they can freely move and are filmed from multiple angles using commercial machine vision cameras. This enables highly accurate 3D reconstruction of key points under the mouse's skin as they freely move around. FIG. 1B displays a graph measuring the pixel intensity of the quantum dots. The pixel intensity lasts for approximately a week within the mouse in the present invention. FIG. 1C shows reflection (top) and fluorescence (bottom) images of the mice injected with quantum dots.

In addition to tracking 3D kinematic trajectories, the system can be used to scale up the training of markerless key point trackers. Two of the most popular methods for tracking key points in animals and humans are SLEAP (Pereira et al., 2022) and DeepLabCut (Mathis et al., 2018). Currently, training these algorithms for a new task requires laborious hand-curated labeling of hundreds if not thousands of frames. In the current invention, millions of frames can be automatically labeled through detection of fluorescence signals originating from injected quantum dots. Such extensive labeling will allow, for the first time, an ability to create foundation models for key point identification in multiple organisms.

With the combination of quantum dots and the 3D pose tracking systems, a comprehensive understanding of the body's true kinematics may be gained, thereby contributing insights into understanding complex motor patterns of rodent models, with implications for movement disorders like Huntington's and Parkinson's disease.

### Methods and Materials

### Quantum Dot Mixtures

QTracker™ 800 Vascular Labels (ThermoFisher Scientific, #Q21070MP) and QTracker™ 800 Cell Labeling Kit (ThermoFisher Scientific #Q25070MP) were used as stocks for the quantum dot injections. For vehicle, 1× borate buffer in 1× phosphate buffered saline (PBS) was used.

### Surgical Procedure

Mice are anesthetized with isoflurane using a nose cone. The procedures are carried out on a heating pad and completed within 30 minutes. The fur over the spine is shaved and treated with Nair. The mixtures are injected using a sterile pulled glass micro-pipette (Drummond 3-000-210-G, diameters that were approximately 0.1 mm) created using a Sutter P-2000 laser puller (parameters: heat 450, filament 4, velocity 150, delay 175, pull 35). The micro-pipette is attached to a modified positive-displacement microinjector (Drummond 3-000-510-X). Specifically, the stainless-steel plunger of the microinjector is cut to enable the pulled micropipette to be safely attached to the microinjector. A volume of 2 μL of the mixture is delivered to each of the 14 injection sites either intra- or sub-dermally: the paws (dorsal and ventral), the tail (base, midsection, and tip), and the back (upper, middle, and lower region). Following each injection, 70% ethanol and triple antibiotic ointment are applied to minimize potential complications. The animals are allowed to recover in their cage for one hour after the procedure.

### Recording Arena

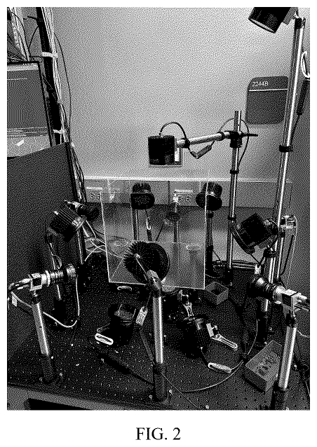

A plexiglass arena is created from clear cast-acrylic panels (McMaster-Carr 8560K184). Interdigitated patterns are cut into the edges of panels to case fitting together. Panels are glued together using acrylic plastic cement (Sci-Grip 10315). Custom 3D-printed molds are secured to the bottom acrylic panel (Torr-Seal Epoxy, Varian), and screw-to-expand brass inserts are inserted into the molds to secure ¼-20″ set screws. The other end of the set screws are inserted into Thorlabs 1″ optical posts to, in turn, secure to an optical breadboard placed on top of a leveled frame (PFM52503). An image of the recording arena can be seen in FIG. 2.

### Recording Hardware

The plexiglass arena is filmed using 5 hardware-synchronized NIR-optimized Basler USB3 cameras (acA2040-90 um). For wide field-of-view imaging the cameras are outfit with a Thorlabs machine vision lens (MVL8M1). In order to prevent imaging of quantum dot excitation light, long-pass (MidOpt LP830-55) and polarization filters (PR1000-55) are secured to the lens. Polarization filters are rotated until excitation light is minimized. For exciting quantum dots at wavelengths compatible with imaging through skin we used NIR-I emitting LED lights (SL246-730IC) outfit with polarization filters (PA371-S82). For collecting reflectance images, IR emitting LED lights are used (SL246-850IC). The lights are triggered in a temporally multiplexed configuration so that fluorescence and reflectance data could collected near-simultaneously using the following sequence: (1) IR lights on for 10 milliseconds (2) all lights off for 1 millisecond (3) NIR lights on for 23 milliseconds, (4) all lights off for 1.5 milliseconds. Power to the LED lights is supplied from a benchtop voltage source (B&K Precision BK1550). Lights are triggered using 5V signals generated from an Arduino Uno, which is also used to trigger camera exposures via the GPIO lines on the Basler cameras. The illumination sequence is repeated for ˜5 minutes for each recording session. In order to capture mice at all angles, the cameras are arranged in a pentagonal formation surrounding the plexiglass arena.

### Recording Software

Camera control and image acquisition software is written in Python. Custom software is also written for the Arduino to trigger the cameras over the appropriate GPIO line along with the LED lights.

### REFERENCES

Mathis, A., Mamidanna, P., Cury, K. M., Abe, T., Murthy, V. N., Mathis, M. W., & Bethge, M. (2018). DeepLabCut: markerless pose estimation of user-defined body parts with deep learning. Nature Neuroscience, 21 (9), 1281-1289.
Monsees, A., Voit, K.-M., Wallace, D. J., Sawinski, J., Charyasz, E., Scheffler, K., Macke, J. H., & Kerr, J. N. D. (2022). Estimation of skeletal kinematics in freely moving rodents. Nature Methods, 19 (11), 1500-1509.
Pereira, T. D., Tabris, N., Matsliah, A., Turner, D. M., Li, J., Ravindranath, S., Papadoyannis, E. S., Normand, E., Deutsch, D. S., Wang, Z. Y., Mckenzie-Smith, G. C., Mitelut, C. C., Castro, M. D., D'Uva, J., Kislin, M., Sanes, D. H., Kocher, S. D., Wang, S. S. H., Falkner, A. L., . . . Murthy, M. (2022). SLEAP: A deep learning system for multi-animal pose tracking. Nature Methods, 19 (4), 486-495.
Reineck, P., & Gibson, B. C. (2017). Near-Infrared Fluorescent Nanomaterials for Bioimaging and Sensing. Advanced Optical Materials, 5 (2), 1600446.

1. A method of measuring fluorescence in an animal, the method comprising (a) injecting the animal with one or more fluorescent particles; and (b) capturing the fluorescence exhibited by the animal.

2. The method of claim 1, wherein the fluorescent particle is a quantum dot.

3. The method of claim 2, wherein the quantum dot emits at an excitation from about 600 nm to about 1000 nm.

4. The method of claim 1, wherein the animal is a mammal.

5. The method of claim 4, wherein the mammal is a mouse.

6. The method of claim 5, wherein the mouse is injected in at least one of the right paw, the left paw, the right hind leg, the left hind leg, the tail, subdermal, or the spine.

7. The method of claim 1, wherein a camera captures the fluorescence.

8. The method of claim 7, wherein the camera is a near infrared camera.

9. The method of claim 1, wherein the animal is a diseased animal.

10. The method of claim 9, wherein the disease is a neurodegenerative disease.

11. The method of claim 10, wherein the neurodegenerative disease is Parkinson's or Huntington's.

12. The method of claim 2, wherein the animal is a mammal.

13. The method of claim 3, wherein the animal is a mammal.

14. The method of claim 2, wherein a camera captures the fluorescence.

15. The method of claim 3, wherein a camera captures the fluorescence.

16. The method of claim 4, wherein a camera captures the fluorescence.

17. The method of claim 5, wherein a camera captures the fluorescence.

18. The method of claim 6, wherein a camera captures the fluorescence.

19. The method of claim 2, wherein the animal is a diseased animal.

20. A method of measuring movement of an animal, the method comprising:
(a) injecting the animal with one or more fluorescent particle(s);
(b) capturing the fluorescence exhibited by the animal; and
(c) monitoring the fluorescence to measure the movement of the animal;
wherein movement of the animal is measured using a camera;
wherein the animal has a neurodegenerative disorder;
wherein the method further comprises comparing the movement of the animal to a set of criteria to determine if the animal suffers from a neurodegenerative disorder; and
wherein the criteria is if the animal has at least one of a halting gait, a tremor, dyskinesia, tics, or chorea.
